(19) United States
(12) Patent Application Publication
(10) Pub. No.: US 2023/0172575 A1
(43) Pub. Date: Jun. 8, 2023

(54) METHOD, SYSTEM AND COMPUTER PROGRAM PRODUCT FOR DETERMINING ISCHEMIA REGION OF THE ORGAN

(71) Applicant: NARODOWY INSTYTUT KARDIOLOGII STEFANA KARDYNALA WYSZYNSKIEGO - PANSTWOWY INSTYTUT BADAWCZY, Warszawa (PL)

(72) Inventors: Cezary KEPKA, Jozefow (PL); Mariusz KRUK, Sulejowek (PL); Anna OLEKSIAK, Warszawa (PL)

(21) Appl. No.: 17/996,833

(22) PCT Filed: Apr. 21, 2021
(86) PCT No.: PCT/PL2021/050028

§ 371 Date: Oct. 21, 2022

(30) Foreign Application Priority Data
Apr. 21, 2020 (PL) P.433612

(51) Int. Cl. A61B 6/00; G06V 10/26; G06V 10/25; G06T 11/00; G16H 30/40

(52) U.S. Cl. CPC A61B 6/5217; A61B 6/503; A61B 6/504; A61B 6/507; G06V 10/26; G06V 10/25; G06T 11/005; G16H 30/40; G06T 2211/404; G06V 2201/031

(57) ABSTRACT

The invention relates to a method for identifying an ischaemic region (Oₙ) of an organ based on anatomical data, wherein the ischaemic region (Oₙ) is 0.2 to 1 part of the stenosed region at risk (O_{z}) downstream of the threshold point (P_{prog}). The size of the ischaemic region (Oₙ) is proportional to the difference between the indicative value at the threshold point (P_{prog}) and at the measuring point (Pₚₒₘ) in the artery. The invention also relates to a system for identifying organ ischaemia, a computer program for identifying organ ischaemia and a computer program product.

The invention relates to a method for identifying an ischaemic region of an organ, a system for identifying an ischaemic region of an organ, a computer program for identifying an ischaemic region of an organ and a computer program product for identifying an ischaemic region of an organ based on arterial flow data, which provides clinically relevant information required for qualification for further surgical treatment.

A network of arteries encompasses the organ like an inverted river system, with the flow directed towards smaller vessels. The individual arteries split into smaller ones and supply the designated regions (volumes) of the organ like such an inverted river supply area. In the following description the term “supply area” corresponds to said region which is the inverse of the commonly understood definition of river supply region. The more distal (downstream, directed towards the end, as the blood flows) sections of the artery, or branches, cover correspondingly smaller regions of the organ corresponding to the location of an artery. For each point, on each artery, an area (a supply area) may be delineated that is supplied by the artery downstream said point, wherein the area comprises the supply area of an artery located directly downstream of a point at the artery, similar to an (inverted) river supply area.

The distribution of pressures in arteries results in flow towards smaller vessels, and thus distal pressures are lower than the pressure at the point where the artery leaves the aorta, with the difference being only minimal in normal vessels. If, due to a pathology, the diameter of the vessel does not match the size of the area supplied, for example as a result of arterial stenosis, said pressure difference increases. The pressure difference between the arterial outlet and the distal point may be gradual for less pronounced, but more extensive stenoses, or greater for tighter localised stenoses. It is assumed, for example for the cardiac arteries, that if the ratio between the pressure at the distal point (in practice downstream of the affected stenosis in the artery) and at the point where the artery leaves the aorta under conditions of maximum flow, the so-called fractional flow reserve (FFR), is less than or equal to 0.80, or less than or equal to 0.75, or less than or equal to 0.89 for the instantaneous wave-free ratio (iFR), this indicates ischaemia of the region supplied by the artery and is the basis for a decision to revascularise the artery.

Myocardial ischaemia is defined herein as a sufficiently important impairment of organ perfusion during hyperaemia (under conditions of minimal microcirculatory resistance) or stress that may be identified using established methods of assessing ischaemia such as: perfusion assessment using stress computed tomography or perfusion assessment using stress magnetic resonance or perfusion assessment using stress single-photon emission computed tomography (SPECT) or positron emission tomography (PET).

Attempts have been documented in the literature to determine, either in mass units, or, alternatively, as a percentage of the total heart mass, the region at risk of ischaemia of the myocardium based on the image of coronary arteries and the location of stenoses (paper “Incremental Value of Subtended Myocardial Mass for Identifying FFR-Verified Ischemia Using Quantitative CT Angiography: Comparison With Quantitative Coronary Angiography and CT-FFR”; doi.org/10.1016/j.jcmg.2017.10.027), where the region of myocardium supplied downstream of the stenosis is referred to for example as the “mass at risk” (region at risk), with the maximum stenoses corresponding to the minimum vessel lumen within the stenosis. In the studies available in the literature, the mass at risk thus defined is identified as an ischaemic region. However, it is not known whether a greater stenosis at a given point results in:
a) more advanced ischaemia of the entire region at risk downstream of the stenosis; or
b) a larger region of ischaemia within the region at risk downstream of the stenosis;
   for b), there are no methods known for identifying the ischaemic region (either relative or absolute) based on anatomical data (image of the heart, coronary arteries, arterial stenoses). The resolution of the problem described above, namely determining whether a) or b) is true, and the potential assessment of the extent of ischaemia in variant b) is crucial for clinical decision-making, e.g., for qualifying patient for surgical treatment of coronary artery disease. It is known that interventional treatment of coronary artery disease improves prognosis and reduces mortality and myocardial infarctions only in patients with severe ischaemia, such as more than 10% or in at least two segments of the left heart ventricle. Identification of ischaemia as such, for example an FFR result less than or equal to 0.80 or an iFR less than or equal to 0.89 provides no feedback on the size of the ischaemic region.

Document JP2018064967AA discloses a medical image processing device, a medical image processing method and a recording medium. In the solution, the occurrence of stenosis, with an associated ischaemic region is determined based on computed tomography (CT) data. The stenosis is then verified by measuring the flow in the vessel and determining values such as FFR. In this solution, the ischaemic region is estimated based on CT perfusion study, i.e., the ischaemic region is identified based on a test with additional contrast administration and additional scanning The results obtained allow for identifying the location to insert a bypass. The solution involves generating 2D and 3D heart images.

Document US20190318476A1 discloses a method and system for assessing vascular blockage based on machine learning. Methods and systems acquire, using coronary CT artery angiography (CCTA) method, a volumetric image dataset for the target organ that encompasses the vessel of interest. They extract the axial trajectory extending along the vessel of interest (VOI) in the volumetric image dataset and yield a three-dimensional (3D) multi-plane structured image based on the volumetric image dataset and the axial trajectory of the VOL Stenoses in vessels are classified accordingly based on the FFR value.

Patent description EP3041414B1 discloses a device, method, system and computer program for processing cardiac data for the purpose of imaging the heart of a living being. The processing device uses FFR values and myocardial perfusion values. The distribution of FFR values is identified based on 3D geometry of the coronary artery tree obtained from computed tomography angiography (CTA), and the perfusion value is determined based on spectral CT measurements. Both values are analysed for a match based on the distribution of FFR values and the distribution of myocardial perfusion values. The match, if found, confirms the reliability of the FFR data and allows for obtaining an accurate heart model. The display of combined FFR and perfusion information, performed as an additional test with contrast and additional scanning, allows the physician to better identify a critical stenosis. The systems and methods described can be used in CT angiography and myocardial perfusion imaging to evaluate patients with low to moderate risk ischaemic heart disease.

On the one hand, methods illustrating the distribution of arterial pressure that assess the pressure gradient across stenoses within an artery, in order to determine the presence or absence of ischaemia caused by the stenosis and to qualify the patient for revascularisation if the stenosis reaches a threshold gradient value are known from prior art. These methods include invasive methods that require additional arterial instrumentation to measure the fractional flow reserve (FFR) or iFR, as well as non-invasive methods based on simulations involving fluid mechanics or predicting (machine learning) pressure distributions based on images of the arteries and their stenoses obtained in a non-invasive test, such as computed tomography angiography (CT-FFR, FFR-CT) or invasive angiography (FFR-QCA).

On the other hand, methods for the so-called perfusion assessment of ischaemia, based on the evaluation of the ischaemic region of an organ, which do not require the knowledge of the anatomy of the coronary arteries, such as SPECT, PET, MRI, contrast echocardiography, which are based on imaging areas without contrast or radiotracer uptake usually under conditions of pharmacological or physiological stress (exercise) in the organ itself, such as the heart are also known. They allow an ischaemic region to be assessed in quantitative absolute terms (mass, volume) and relative terms (percentage of the volume or mass of the entire organ).

Using various techniques (either invasive or non-invasive), it is possible to determine the distribution of blood pressure in the arteries of various organs, such as the heart, taking into account the presence of arterial stenoses, and to identify at which point in the artery the pressure ratio between a particular point and the point where the artery leaves the aorta reaches the threshold value sufficient for the diagnosis of ischaemia. However, no methods are known for accurately identifying the region of organ ischaemia within the region at risk.

A method for identifying an ischaemic region of an organ based on anatomical data, comprising a step of acquiring data on the arterial tree of the organ and the shape of the organ and a step of identifying a threshold point in the artery at which a threshold indicative value is reached that corresponds to the ischaemia of the organ characterised in that it comprises the steps of:
acquiring data on the arterial tree of the organ and the shape of the organ; extracting arterial vessels of the organ and identifying the volume of the organ;
identifying a threshold point in an artery at which a threshold indicative value is reached that corresponds to the ischaemia of the organ and identifying the indicative value in a particular artery at the measuring point situated downstream of the threshold point;
qualifying an artery downstream of the threshold point of the stenosed region at risk being supplied;
identifying a stenosed region at risk;
calculating the volume of the ischaemic region as part of the stenosed region at risk. The ischaemic region is 0.2-1 part of the stenosed region at risk downstream of the threshold point, and the size of the ischaemic region is proportional to the difference between the indicative value at the threshold point and at the measuring point. The method then involves superimposing onto the image of the organ, acquired in the step of acquiring data, the ischaemic region of the organ identified in the step of calculating.

Preferably, the step of identifying the arterial vessels of the organ and the volume of the organ is followed by the step of identifying the pressure distribution in the tested arteries relative to the pressure at a reference point.

Preferably, the ischaemic region is located at the most distal point relative to the threshold point in the artery.

Preferably, the measuring point is approx. 20 mm downstream of the threshold point.

Preferably, the ischaemic region represents 0.5 part of the stenosed region at risk downstream of the threshold point plus a ratio score of 0.1 to 0.05 of the difference between the indicating value at the threshold point and the indicating value at the measurement point.

Preferably, image data are acquired in the step of acquiring data.

Preferably, the step of acquiring data on the arterial tree and the shape of the target organ is performed by computed tomography angiography.

Preferably, the step of acquiring data on the arterial tree is performed by invasive angiography, and the shape of the target organ is reconstructed based on said data.

Preferably, the step of identifying the pressure distribution in the tested arteries is performed using an actual measurement.

Preferably, the step of identifying the pressure distribution in the tested arteries is performed using digital methods such as computer simulation.

Preferably, the reference point is the arterial outlet from the aorta or the reference point is the aortic sac.

Preferably, the indicative value is the flow fractional reserve value, and the threshold indicative value of the flow fractional reserve is equal to or less than 0.8.

Preferably, the flow fractional reserve value is obtained based on a computed tomography scan or a computer simulation.

Preferably, the indicative value is the ratio of the pressure at the threshold point to the pressure at the arterial outlet or the indicative value is the ratio of the pressure at the measuring point to the pressure at the arterial outlet.

Preferably, the step of qualifying an artery downstream of a threshold point of a stenosed region at risk is performed using at least one method from: a Voronoi diagram, a stem-and-crown model, an American Heart Association diagram.

Preferably, the step of identifying a stenosed region at risk is performed using a quantitative analysis of the volume of the stenosed region at risk.

Preferably, the step of identifying a stenosed region at risk is performed using a percentage analysis relative to total organ volume of the stenosed region at risk.

Preferably, the superimposing step is performed using a Voronoi diagram to visualise the ischaemic region.

A system for identifying ischaemic region of an organ based on anatomical data comprises a module for acquiring data on the arterial tree of the organ and the shape of the organ, and a module for identifying the threshold point in the artery at which the threshold indicative value corresponding to organ ischaemia is reached. The system comprises:
a module for extracting arterial vessels of an organ and identifying the volume of an organ;
a module for identifying a threshold point in an artery at which a threshold indicative value is reached that corresponds to the ischaemia of the organ and a module for identifying the indicative value in a particular artery at the measuring point situated downstream of the threshold point;
a module for qualifying an artery downstream of the threshold point of the stenosed region at risk being supplied;
a module for identifying a stenosed region at risk;
a module for calculating the volume of the ischaemic region as a part of the stenosed region at risk, wherein the ischaemic region is 0.2-1 part of the stenosed region at risk downstream of the threshold point, wherein the size of the ischaemic region is proportional to the difference between the indicative value at the threshold point and at the measuring point; and
a module for superimposing the ischaemic region of an organ onto the image of the organ acquired by the module for acquiring data.

Preferably, the system further comprises a module for identifying the pressure distribution in the tested arteries, relative to the pressure at a reference point, located downstream of the module for identifying the arterial vessels of the organ and the volume of the organ.

Preferably, the module for acquiring data acquires image data.

Preferably, the module for acquiring data on the arterial tree and the shape of the target organ is performed by computed tomography angiography.

Preferably, the module for acquiring data on the arterial tree uses invasive angiography, and the shape of the target organ is reconstructed based on said data.

Preferably, the module for identifying the pressure distribution in the tested arteries uses actual measurements or uses digital methods, wherein preferably, the digital method is computer simulation.

Preferably, the module for qualifying an artery downstream of a threshold point of a stenosed region at risk uses at least one method from: a Voronoi diagram, a stem-and-crown model, an American Heart Association diagram.

Preferably, the module for identifying a stenosed region at risk uses quantitative analysis of the volume of the stenosed region at risk or uses a percentage analysis relative to the total organ volume of the stenosed region at risk.

Preferably, the superimposing module uses a Voronoi diagram to visualise the ischaemic region.

A computer program for identifying organ ischaemia, comprising instructions for performing the method according to any one of the steps of the method for identifying an ischaemic region of an organ.

A product of a computer program for identifying organ ischaemia, comprising a computer readable code performing the steps of the method according to any one of the steps of the method for identifying an ischaemic region of an organ.

The method according to the invention can be used to quantify the ischaemic region of an organ, such as the heart, based on recorded or simulated pressure distributions in the vessel tested and anatomical/geometrical data, i.e., images or simulated images of the organ tested. The added value of the method is the information on the size of organ ischaemia, which could constitute an important addition to the qualification for surgical treatment, such as of coronary artery disease.

According to the invention, the ischaemic region of an organ, such as the heart, extends in the supply area of a given artery downstream of the point where the pressure distribution reaches a threshold value diagnostic of ischaemia of the given organ, such as a value of 0.80 or 0.75 for invasive FFR measurement, FFR based on flow simulation or machine learning based on images of computed tomography of coronary arteries (CT FFR), or invasive angiography (QCA-FFR).

The method is innovative, as it introduces a new distinction, which is crucial for appropriate treatment, between the traditionally understood region at risk of ischaemia, i.e., downstream of the stenosis, and the stenosed region at risk downstream of the point where the value of pressure change exceeds the threshold value diagnostic of ischaemia of the organ in question, and the ischaemic region. Surprisingly, the Applicant found that in many cases the ischaemic region is much smaller than the region at risk, understood as both the region downstream of the stenosis and the region downstream of the point where the value of the pressure change exceeds the threshold value diagnostic of ischaemia of the organ in question (stenosed region at risk). The method allows for identifying the region of ischaemia of the organ tested, which, for example in the case of the heart, defines the indications for treatment by coronary revascularisation. Due to the anticipated widespread use of the virtual FFR in clinical practice, forming the basis of the described method, the solution according to the invention may be widely applicable. Additional analysis and identification of a smaller region of ischaemia may reduce the number of revascularisation procedures that will not be advantageous for the patient in terms of reducing the risk of myocardial infarction/death.

Approximately 5 million coronary revascularisation procedures are performed annually worldwide, and the trend is growing. If the recommendations of the cardiology societies regarding indications for revascularisation were followed, some of these procedures could be avoided. The method according to the invention can potentially reduce the indications for procedures given that, as regards stenoses in proximal sections of large vessels, it may indicate a smaller ischaemic region than is currently assumed. Showing the benefits of revascularisation in clinical trials only in patients with severe ischaemia as assessed by the method as above could result in the method as above being added to the guidelines.

In summary, the solution has the advantage of providing additional information that is clinically important for qualifying patients with arterial stenosis for further treatment.

The present description uses the following definitions:
anatomical data—data comprising information on body structures, parts thereof and organs;
imaging data—data comprising information on body structures, parts anatomical and organs acquired by way of imaging test;
threshold point—a point along an artery at which an analysed physiological parameter assessing the efficiency of the coronary circulation first reaches from the arterial outlet the threshold value allowing for the diagnosis of organ ischaemia; for a coronary artery (heart), for example, for fractional flow reserve (FFR) the threshold value is 0.80 or 0.75; for diastolic pressure gradient (iFR) analysis, the threshold value is 0.89 or 0.86; for coronary reserve, the threshold value is 2.0;
indicative value—the value of a parameter for a specific point in an artery which meets selected physiological parameters relating to the efficiency of the coronary circulation;
indicative value corresponding to organ ischaemia—the value of a parameter analysing physiological parameters assessing the efficiency of the coronary circulation, for which a value diagnostic of organ ischaemia is reached; for example, for the heart the fractional flow reserve (FFR) is equal or less than 0.80 or 0.75; for the diastolic pressure gradient (iFR) analysis, the threshold value is equal or less than 0.89 or 0.86, and for the coronary reserve it is equal or less than 2.0;
measuring point—a point along the artery located distally, such as 20 mm from the threshold point;
identification of a stenosed region at risk—identification of the region (volume) of an organ supplied by a given artery downstream of the threshold point located on said artery;
superimposing on an image—visualisation of the computation results of the ischaemic region in the form of a marking (such as by colour) of the region corresponding to the calculated volume of the ischaemic organ within the stenosed region at risk, in a shape corresponding to the blood supply region of a given artery downstream of the hypothetical point on the artery to which the ischaemia region as above corresponds;
distal—defines the location along the artery from the starting point, i.e., the outlet of the artery from a larger vessel (such as the aorta), towards the circumference of the vessel. “Distally” means “towards the circumference of the vessel”;
actual measurement—means taking measurements such as of coronary flow parameters, using physical methods, such as a measurement with a catheter provided with a pressure sensor, or a Doppler sensor, or a temperature sensor;
digital methods—methods of in silico simulation of physical or physiological processes occurring in the body, such as analysis of fractional flow reserve based on angiography of the coronary arteries in computed tomography;
computer simulation—simulation of physical or physiological processes in silico;
quantitative analysis—analysis of the tissue volume of an organ;
percentage analysis—analysis of the tissue volume of an organ in the region of interest relative to the volume of the organ;
methods for identifying supply areas of an organ by an artery: Voronoi method, stem-and-crown model, patient-specific American Heart Association diagram for identifying coronary artery supply area;
Voronoi algorithm (method) is a mathematical method that divides space by the shortest path to a reference point. The spaces surrounding for example three coronary arteries are gradually expanded until another coronary territory is encountered, followed by termination of the expansion, and the region is then covered by the territory of the corresponding coronary artery (European Radiology volume 27, pages 4044-4053 (2017));
stem-and-crown model—based on allometric scaling between the length of the arterial tree and the mass of the organ;
Allometric scaling law—logarithmic correlation between size, function and energy expenditure in life sciences J Am Coll Cardiol Intv 9:1548-1560;
patient-specific American Heart Association diagram for identifying a coronary artery supply region—superimposition of regions based on the Voronoi method onto the American Heart Association heart territory diagram (Journal of Cardiovascular Magnetic Resonance volume 11, Article number: P103 (2009));
methods for identifying ischaemia based on the measurement/simulation of arterial flows: fractional flow reserve, flow reserve, iFR;
Fractional flow reserve (FFR) is a technique used in catheterisation of e.g., coronary vessels to measure pressure differences upstream and downstream of a coronary artery stenosis in order to determine the probability that the stenosis hinders sufficient blood supply to the myocardium (myocardial ischaemia). Fractional flow reserve is defined as the pressure downstream (distal) of the stenosis relative to the pressure at the arterial outlet from the aorta. The result is an absolute number; an FFR ratio of 0.80 means that a stenosis in question causes a 20% drop in blood pressure;
iFR—is a diagnostic tool used to assess whether a stenosis causes reduced blood flow in the coronary arteries resulting in ischaemia. The iFR is measured during cardiac catheterisation (angiography) using invasive coronary probes that are placed in the coronary arteries to be evaluated (Int J Cardiol Heart Vasc. 2018 Mar. 18: pages 39-45);
Flow reserve is the maximum increase in blood flow through the coronary arteries above the normal resting volume. Its measurement is often used in medicine to help treat conditions affecting the coronary arteries and to determine the effectiveness of treatments administered (Int J Cardiol Heart Vasc. 2018 Mar; 18: pages 39-45).

The object of the invention is illustrated in the drawing, where:

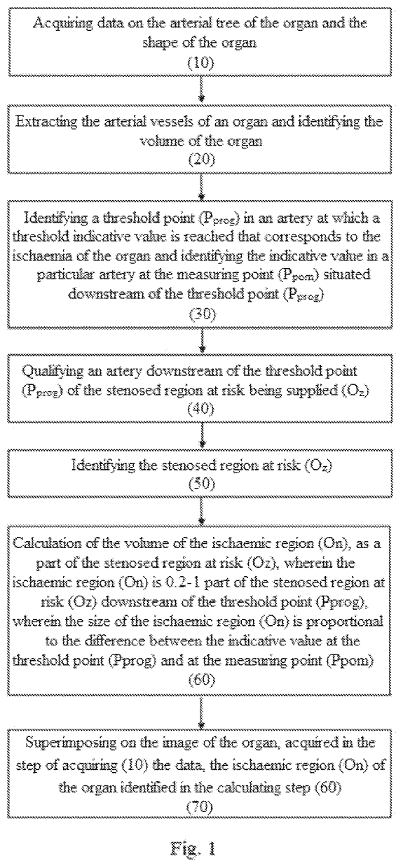

FIG. 1 is a flow chart of the method for identifying an ischaemic region of an organ;

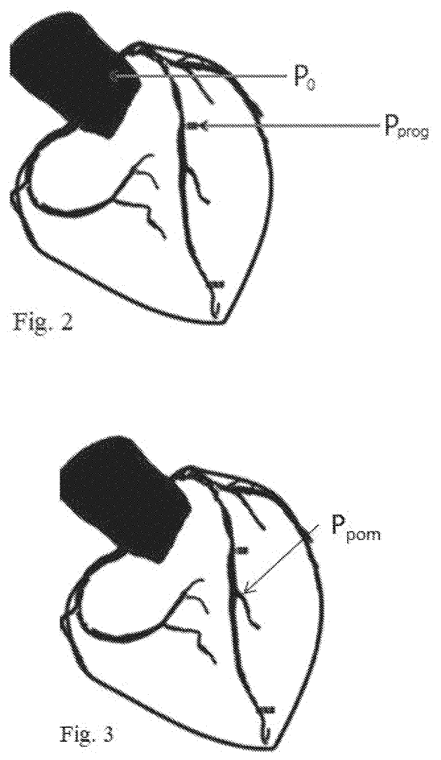

FIG. 2 is a spatial image of the heart with the vascular tree indicating the threshold point in the artery where the threshold value corresponding to organ ischaemia is reached;

FIG. 3 is a spatial image of the heart indicating the measuring point located downstream of the threshold point;

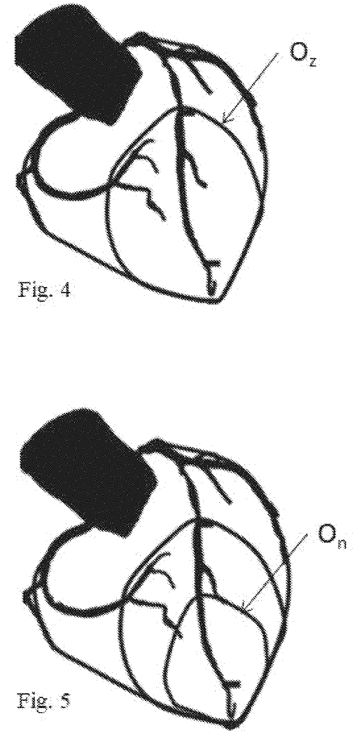

FIG. 4 is a spatial image of the heart indicating the stenosed region at risk, supplied by the artery downstream of the point where the FFR reaches≤0.80;

FIG. 5 is a spatial image of the heart showing the ischaemic region against the stenosed region at risk, wherein the ischaemic region makes part of the stenosed region at risk and extends in the distal direction of the artery from a point which is no more upstream than where the FFR reaches≤0.80;

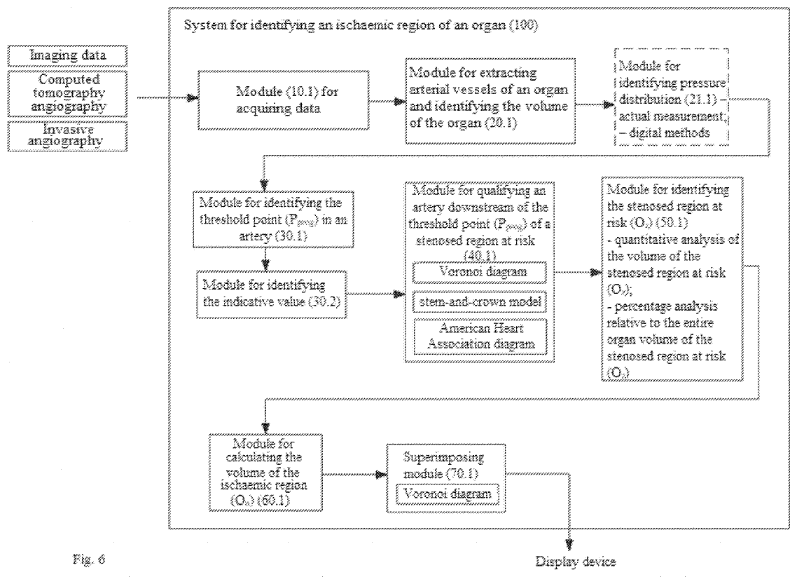

FIG. 6 is a block diagram of the system for identifying organ ischaemia.

In the embodiment illustrated in FIGS. 1-5, the method for identifying an ischaemic region Oₙ of an organ based on anatomical data includes a step of acquiring 10 data on the arterial tree of the organ and the shape of the organ. In an embodiment, the acquired data may be imaging data acquired for example by computed tomography, while in a further embodiment arterial tree data is acquired by way of invasive angiography, and the shape of the target organ is reconstructed based on such pre-acquired data.

The method then comprises extracting 20 the arterial vessels of the organ and identifying the volume of the organ. In another embodiment, the method also includes determining 21 the pressure distribution in the tested arteries, relative to the pressure at a reference point P₀, wherein the reference point P₀ is the point where the artery outlets from aorta, and in a further embodiment it is the aortic sac. The step of identifying 21 the distribution of pressures in the tested arteries is performed by means of actual measurement, and in another embodiment by digital methods such as computer simulation.

Then, following the step of extracting 20 the arterial vessels and optionally following the optional step of identifying 21 the pressure distribution, the method for identifying an ischaemic region Oₙ comprises identifying 30 the threshold point P_{prog} in the artery at which the threshold indicative value corresponding to organ ischaemia is reached, and the method comprises identifying the indicative value in the respective artery at the measuring point Pₚₒₘ, such as approx. 20 mm downstream of the threshold point P_{prog}. In an embodiment, the indicative value corresponding to organ ischaemia is the fractional flow reserve, FFR, which has a threshold value equal to 0.8 or less. The FFR value is obtained based on the actual measurement and, in another embodiment, the FFR value is obtained from a computer simulation based on a computer tomography scan or invasive angiography scan. In another embodiment, the indicative value corresponding to organ ischaemia is the ratio of the pressure (or simulated pressure) at the threshold point P_{prog} to the pressure (or simulated pressure) at the arterial outlet, and in the following example, the indicative value is the ratio of the pressure (or simulated pressure) at the measuring point Pₚₒₘ to the pressure (or simulated pressure) at the arterial outlet.

The subsequent step in the method is the step of qualifying 40 an artery downstream of the threshold point P_{prog} of the stenosed region at risk O_{z} being supplied, for example using the Voronoi diagram/method.

The method then comprises identifying 50 the stenosed region at risk O_{z}, for example using a quantitative analysis of the volume of the stenosed region at risk O_{z}, and in another embodiment by means of a percentage analysis relative to the total organ volume of the stenosed region at risk O_{z}.

Moreover, the present method involves calculating 60 the volume of the ischaemic region Oₙ as a part of the stenosed region at risk O_{z}. The ischaemic region On is located most distal to the threshold point P_{prog} in the artery and represents 0.2-1 part of the stenosed region at risk O_{z} downstream of the threshold point P_{prog}. The size of the ischaemic region Oₙ is proportional to the difference between the indicative value at the threshold point P_{prog} and at the measuring point Pₚₒₘ. In an embodiment, the ischaemic region Oₙ represents 0.5 part of the stenosed region at risk O_{z} downstream of the threshold point P_{prog} plus a ratio score of 0.1 to 0.05 of the difference between the indicative value at the threshold point P_{prog} and the indicative value at the measuring point Pₚₒₘ.

The final step of the present method is the step of superimposing 70 onto the image of the organ, acquired in the step of acquiring 10 data, the ischaemic region Oₙ of the muscle identified in the step of calculating 60. In one embodiment, the step of superimposing 70 is performed using a Voronoi diagram to visualise the ischaemic region Oₙ.

In another embodiment, the method can be used in practice as an additional imaging module overlay, such as CT-FFR, FFR-CT, FFR-QCA, invasive FFR, iFR.

For methods based on computed tomography (CT), the image of the heart acquired during the scan allows for accurate determination of its dimensions and the supply area of the individual arteries. Here, the method can further involve marking and identifying the ischaemic muscle including a calculation of its mass relative to that of the total organ. For FFR-QCA, or an invasive measurement such as FFR or iFR, in order to simulate an ischaemic region Oₙ, it is first required to create a virtual image of the left ventricle (based on ventriculography or the distribution of the coronary arteries alone) and then only to mark and identify the ischaemic muscle in the manner described above.

In another embodiment, the data of a CT coronary artery angiography scan in the form of DICOM files encoding spatial information on the shape and volume of the heart, including the myocardium and coronary arteries and their mutual spatial arrangement were transferred via external medium or network transfer to a station of a computational unit which:
1. Extracts, for example using the software programme “cFFR version 1.4, Siemens Healthcare GmbH”, a three-dimensional image, i.e., it performs an image segmentation process which yields an image of the coronary arteries and myocardium which allows e.g., for visualising the course of the coronary arteries and identifying the location of coronary artery stenosis;
2. In the next step, for example using the programme “cFFR version 1.4, Siemens Healthcare GmbH”, the computational unit determines the pressure distributions in the coronary vessel tree based on the assumptions for the calculation of the simulated fractional flow reserve, FFR. This calculates the ratio of the pressure at a given point along the artery to the pressure where the artery outlets from the aorta, which calculation is shown as colour maps within the arteries, wherein lower distal pressure is tantamount to lower FFR;
3. The computational unit then analyses the individual FFR values along the artery, allowing to identify the threshold point P_{prog} on the stenosed coronary artery, wherein the FFR value is equal to the threshold value (0.80), and to determine the FFR value at the point Pₚₒₘ, which is located 20 mm downstream of the threshold point P_{prog} at 0.76;
4. The imaging data from the same test were read using another software, such as CT Coronary Territories from Ziosoft, which, after marking the course of the coronary arteries and marking the contour of the left ventricle using the Voronoi method, allows for ascribing the cardiac blood supply region expressed in mm³, or as % of the total, to each indicated point on each artery;
5. A threshold point P_{prog} was identified on the stenosed artery, and the computational unit, using the Voronoi method, determined and then superimposed, onto the left ventricular image, the area of cardiac blood supply downstream of this point. It then determined its volume in mm³, or % of the total organ, i.e., identified the stenosed region at risk of O_{z}. The result is 35% of myocardial volume;
6. Based on the magnitude of the difference in FFR values between the threshold point P_{prog} and the measuring point Pₚₒₘ of 0.04, the resulting volume was converted using the following formula:

volume of ischaemic region=(0.5+0.08)*O_{Z}%,

where a factor of 0.58 was obtained as follows: the primary factor of 0.50 increases proportionally by 0.1 part for each 0.05 increase in the FFR value at the measuring point Pₚₒₘ. Since the difference in FFR values at the threshold point P_{prog} and at the measuring point Pₚₒₘ is 0.04, this means an increase of 0.08 in the primary factor to give a score of 20%. Then, in order to visualise the ischaemic region Oₙ, a point was sought on the stenosed artery for which the supply area corresponds to the calculated volume of the ischaemic region Oₙ. After marking this point using the Voronoi method, the software, such as CT Coronary Territories from Ziosoft identifies and displays the ischaemic region Oₙ.

In the embodiment wherein the step of identifying 20 the arterial vessels of an organ and the volume of an organ is followed by the step of identifying 21 the pressure distribution in the tested arteries, relative to the pressure at the reference point P₀, the reference point P₀ is identified at the arterial outlet from the aorta. The pressure distribution can be determined for example using computer simulation of the pressures, such as in the software “cFFR version 1.4, Siemens Healthcare GmbH”, or in conditions of actual measurement with a coronary artery pressure probe, such as Verrata® Pressure Guide Wire, placed at a given point in the artery, and the pressure at a reference point P₀ identified at a guiding catheter, whose end is located at the arterial outlet.

In an embodiment wherein the step of identifying 50 a stenosed region at risk O_{z} is performed by quantifying the volume of the stenosed region at risk O_{z}, the myocardial volume is determined by visually identifying and extracting the myocardium based on three-dimensional images of the heart acquired using computer tomography, or by extraction using software such as CT Coronary Territories from Ziosoft.

The Applicant conducted an experiment in which 34 patients underwent coronary CT scanning, CT-FFR scanning/simulation, and CT (computed tomography) cardiac perfusion scanning using a vasodilator (adenosine or regadenoson). Based on the results, a reference ischaemic region Oₙ of the heart expressed as % of total myocardial mass was identified. In the patients studied, a number of points were marked on the stenosed coronary artery starting from the point of maximum stenosis (minimal lumen), in the distal direction of the artery, for which the corresponding “regions at risk” and the corresponding CT-FFR values were recorded. The results obtained were subjected to a proper analysis, which showed the following:
there was no significant correlation between the percentage (%) area of actual ischaemia in the reference study and the “region at risk” expressed as % of myocardial mass marked on the stenosed coronary artery at the point of maximum stenosis. This result indicates that the region of actual ischaemia Oₙ differs significantly (is not the same as) from the “region at risk downstream of the point of maximum stenosis”;
a significant correlation between the ischaemic region Oₙ in the reference study and the correlation derived from the % value of the stenosed muscle region identified downstream of the point where the (CT)FFR value reaches the diagnostic value of ischaemia (0.80 for the heart), the so-called stenosed region at risk O_{z}. Correlations are both quantitative (% of the muscle in the reference method correlated with calculated % of the muscle) and qualitative (large/substantial ischaemic region On of the heart (>=10%)).

As an example, the results obtained show that the % (percentage) of the ischaemic muscle is equal to the % (percentage) of the stenosed area at risk O_{z} of the muscle (defined at the point where CT FFR assumes the value of 0.75) multiplied by a factor of 0.62 and to which a constant of 2.9 is added. In yet another embodiment, the % (percentage) of the ischaemic muscle is equal to the % (percentage) of the stenosed region at risk O_{z} of the muscle (defined at the point where CT FFR assumes the value 0.80) multiplied by a factor of 0.51 and to which a constant of 2.1 is added. In yet another embodiment, the % (percentage) of ischaemic muscle is equal to the averaged % (percentage) of the stenosed region at risk O_{z} of the muscle for a number of points, divided by the averaged FFR (CT FFR or iFR) value for said points (for values below the ischaemia threshold) multiplied by a factor of 0.49 to which a constant of 1.4 is added.

It generally follows from the embodiments above that the percentage of the ischaemic region Oₙ is on average a constant 0.50 (range 0.2-1) multiplied by the percentage (volume) of the stenosed muscle region, i.e., the stenosed region at risk O_{z}, identified for the point at which the FFR (or CT FFR or iFR) value varying along the vessel first takes at least the threshold value for the value diagnostic of organ ischaemia (for the heart≤0.80 or iFR≤0.89). Also, the volume of ischaemic muscle increases as the dynamics of the decrease in FFR (or CT FFR or iFR) value, i.e., the lower the FFR/CTFFR/iFR at the measuring point Pₚₒₘ, the greater the percentage of ischaemic region Oₙ represents relative to the stenosed region at risk O_{z} identified downstream of the threshold point P_{prog}. The location of the ischaemic muscle represents the region having the volume (%) as calculated above and located most distally in the stenosed region at risk O_{z}.

In the embodiments, the Applicant has assumed that significant ischaemia is a region with at least 10% ischaemia, such as a stenosed region at risk O_{z} of muscle>11% downstream of the point for which the FFR (or CT FFR) is 0.75, or a stenosed region at risky O_{z} of muscle>17% downstream of the point for which the FFR (or CT FFR) is 0.80 (FIG. 4).

In another embodiment, all of the steps of the method for identifying an ischaemic region Oₙ of an organ as described above may be performed by a computer programme for identifying ischaemia of an organ comprising instructions for performing said steps. In a further embodiment, all of the steps of the above-described method for identifying an ischaemic region Oₙ of an organ can be performed using a computer programme product comprising a computer readable code.

As shown in FIG. 6, the system 100 for identifying an ischaemic region Oₙ of an organ based on anatomical data, comprises a module 10.1 for acquiring data on the arterial tree of the organ and the shape of the organ, and a module 30.1 for identifying the threshold point P_{prog} in the artery at which the threshold indicative value corresponding to organ ischaemia is reached. The system 100 also comprises:
module 20.1 for extracting arterial vessels of an organ and identifying the volume of an organ;
module 30.1 for identifying a threshold point P_{prog} in an artery at which a threshold indicating value is reached that corresponds to the ischaemia of the organ and a module 30.2 for identifying the indicative value in a particular artery at the measuring point Pₚₒₘ situated downstream of the threshold point P_{prog};
module 40.1 for qualifying an artery downstream of the threshold point P_{prog} of the stenosed region at risk O_{z} being supplied;
module 50.1 for identifying a stenosed region at risk O_{z};
module 60.1 for calculating the volume of the ischaemic region Oₙ as a part of the stenosed region at risk O_{z}, wherein the ischaemic region Oₙ is 0.2-1 part of the stenosed region at risk O_{z} downstream of the threshold point P_{prog}, wherein the size of the ischaemic region Oₙ is proportional to the difference between the indicative value at the threshold point P_{prog} and at the measuring point Pₚₒₘ; and
a module 70.1 for superimposing the ischaemic region Oₙ of an organ onto the image of an organ acquired by the module 10.1 for acquiring data.

In another embodiment, the system 100 further comprises module 21.1 for identifying the pressure distribution in the tested arteries relative to the pressure at a reference point P₀, located downstream of the module 20.1 for identifying the arterial vessels of the organ and the volume of the organ.

In further embodiments of the system 100, module 10.1 for acquiring data on the arterial tree and target organ shape data acquires image data.

In other embodiments, module 10.1 for acquiring data on the arterial tree and target organ shape data uses computed tomography angiography or uses invasive angiography, wherein the target organ shape is reconstructed based on said data.

In another embodiment of the system 100, module 21.1 for identifying the pressure distribution in the tested arteries uses actual measurements.

In another embodiment of the system 100, module 21.1 for identifying the pressure distribution in the tested arteries uses digital methods, such as computer simulation.

In further embodiments of the system 100, module 40.1 for qualifying an artery downstream of a threshold point P_{prog} of a stenosed area at risk uses at least one method from: a Voronoi diagram, a stem-and-crown model, an American Heart Association diagram.

In another embodiment of the system 100, module 50.1 for identifying a stenosed region at risk O_{z} uses a quantitative analysis of the volume of the stenosed region at risk O_{z}, and in a further embodiment, module 50.1 for identifying the stenosed region at risk O_{z} uses a percentage analysis to the total organ volume of the stenosed region at risk O_{z}.

In another embodiment of the system 100, the superimposing module 70.1 uses the Voronoi diagram to visualise the ischaemic region On.

In one embodiment, the system 100 according to the invention is implemented on a processor in any server or PC type computing system.

In another embodiment, the system 100 comprises a processor and a memory for storing instructions that is coupled to the processor, wherein the execution of the instructions by the processor causes the processor to perform the steps of the above-described method for identifying an ischaemic region. The processor may be suitably configured to cause the software modules to perform the steps of the method for identifying an ischaemic region.

All embodiments of the method for identifying an ischaemic region of an organ Oₙ also refer to a system for identifying an ischaemic region of an organ, a computer program for identifying an ischaemic region of an organ, and a computer program product.

Each of the blocks of the diagram illustrating the method for identifying an ischaemic region and each of the blocks of the diagram of the system for identifying an ischaemic region can be implemented by computer program instructions. Such instructions may be provided to a processor of a general-purpose computer, a special purpose computer or another programmable data processing device, such that the instructions that are executed by the processor of the computer or another programmable data processing device allow for implementing the functions as defined in the method and system diagrams.

Aspects of the present invention may be implemented by a computer or devices such as a CPU (central processing unit) or MPU (memory protection unit) which read and execute a computer program product stored in a storage device for performing the functions of the embodiments described above. Aspects of the present invention may also be implemented by a method whose steps are performed by a computer of the system or device, such as by reading and executing a program stored on a storage device for performing the functions of the above-described embodiments. Accordingly, the computer program product is delivered to a computer, for example, via a network or another storage medium used as a storage device. The computer program product according to the invention further comprises a non-volatile machine-readable medium.

Embodiments herein are provided only as non-limiting guidelines for the invention and by no means do they limit the scope of protection as defined by the claims. It should be noted that any technical solution used in the invention can be implemented using equivalent technologies without departing from the scope of protection.

1-37. (canceled)

38. A method for identifying an ischaemic region (Oₙ) of a heart based on anatomical data, comprising:
acquiring data on the arterial tree of the heart and the shape of the heart;
identifying a threshold point (P_{prog}) in an artery at which a threshold indicative value is reached that corresponds to the ischaemia of the heart;
acquiring data on the arterial tree of the heart and the shape of the heart;
extracting the arterial vessels of a heart and identifying the volume of the heart;
identifying a threshold point (P_{prog}) in the artery at which a threshold indicative value is reached that corresponds to the ischaemia of the heart, and identifying the indicative value in a particular artery at the measuring point (Pₚₒₘ) situated downstream of the threshold point (P_{prog});
qualifying an artery downstream of the threshold point (P_{prog}) of the stenosed region at risk (O_{z}) being supplied;
identifying the stenosed region at risk (O_{z});
calculating the volume of the ischaemic region (Oₙ) as a part of the stenosed region at risk (O_{z}), wherein the ischaemic region (Oₙ), located at the most distal point relative to the threshold point (P_{prog}) in the artery, represents 0.5 part of the stenosed region at risk (O_{z}) downstream of the threshold point (P_{prog}) plus the ratio score of 0.1 to 0.05 of the difference between the indicative value at the threshold point (P_{prog}) and the indicative value at the measuring point (Pₚₒₘ); and
superimposing onto the image of the heart, acquired in the step of acquiring data, the ischaemic region (Oₙ) of the heart identified in the step of calculating.

39. The method according to claim 38, wherein the step of identifying the arterial vessels of the heart and the volume of the heart is followed by the step of identifying the pressure distribution in the tested arteries relative to the pressure at a reference point (P₀).

40. The method according to claim 38, wherein the measuring point (Pₚₒₘ) is located approximately 20 mm downstream of the threshold point (P_{prog}).

41. The method according to claim 38 wherein image data is acquired at the step of acquiring data, and wherein the step of acquiring data on the arterial tree and the shape of the target organ is performed by one of computed tomography angiography or invasive angiography and when the step of acquiring data is based on invasive angiography, the shape of the target organ is reconstructed based on said data.

42. The method according to claim 39, wherein the step of identifying the pressure distribution in the tested arteries is performed using an actual measurement or digital computer simulation methods.

43. The method according to claim 39, wherein the reference point (P₀) is the artery outlet from the aorta or the aortic sac.

44. The method according to claim 38, wherein the indicative value is one of the fractional flow reserve (FFR) value, the ratio of the pressure at the threshold point (P_{prog}) to the pressure at the arterial outlet, or the ratio of the pressure at the measuring point (Pₚₒₘ) to the pressure at the arterial outlet.

45. The method according to claim 44, wherein the threshold value indicative of the fractional flow reserve (FFR) is equal to or less than 0.8.

46. The method according to claim 44, wherein the fractional flow reserve (FFR) value is obtained based on a computed tomography scan or a computer simulation.

47. The method according to claim 38, wherein the step of qualifying (40) an artery downstream of a threshold point (P_{prog}) of a stenosed region at risk is performed using at least one method from: a Voronoi diagram, a stem-and-crown model, an American Heart Association diagram.

48. The method according to claim 38, wherein the step of identifying a stenosed region at risk (O_{z}) is performed by a quantitative analysis of the volume of the stenosed region at risk (O_{z}) or by a percentage analysis relative to the total organ volume of the stenosed region at risk (O_{z}).

49. The method according to claim 38, wherein the superimposing step is performed using a Voronoi diagram to visualise the ischaemic region (Oₙ).

50. A system for identifying an ischaemic region (Oₙ) of a heart based on anatomical data, comprising:
a module for acquiring data on the arterial tree of the heart and the shape of the heart;
a module for identifying the threshold point (P_{prog}) in an artery at which the threshold indicative value corresponding to heart ischaemia is reached:
a module for extracting arterial vessels of a heart and identifying the volume of a heart;
a module for identifying a threshold point (P_{prog}) in the artery at which a threshold indicative value is reached that corresponds to the ischaemia of the heart and a module (30.2) for identifying the indicative value in a particular artery at the measuring point (Pₚₒₘ) situated downstream of the threshold point (P_{prog});
a module for qualifying an artery downstream of the threshold point (P_{prog}) of the stenosed region at risk (O_{z}) being supplied;
a module for identifying a stenosed region at risk (O_{z});
a module for calculating the volume of the ischaemic region (Oₙ) as a part of the stenosed region at risk (O_{z}), wherein the ischaemic region (Oₙ), located at the most distal point relative to the threshold point (P_{prog}) in the artery, represents 0.5 part of the stenosed region at risk (O_{z}) downstream of the threshold point (P_{prog}) plus the ratio score of 0.1 to 0.05 of the difference between the indicative value at the threshold point (P_{prog}) and the indicative value at the measuring point (Pₚₒₘ); and
a module for superimposing the ischaemic region (Oₙ) of a heart onto the image of a heart acquired by the module for acquiring data.

51. The system according to claim 50, wherein the system further comprises a module for identifying the pressure distribution in the tested arteries relative to the pressure at a reference point (P₀), located downstream of the module for identifying the arterial vessels of the heart and the volume of the heart.

52. The system according to claim 50, wherein the module for acquiring data acquires image data and wherein the module for acquiring data on the arterial tree and the shape of the target organ uses computed tomography angiography or the module for acquiring data on the arterial tree uses invasive angiography, and the shape of the target organ is reconstructed based on said data.

53. The system according to claim 50, wherein the module for identifying the pressure distribution in the tested arteries uses actual measurements or digital computer simulation methods.

54. The system according to claim 50, wherein the module for qualifying an artery downstream of a threshold point (P_{prog}) of a stenosed region at risk uses at least one method from: a Voronoi diagram, a stem-and-crown model, an American Heart Association diagram.

55. The system according to claim 50, wherein the module for identifying a stenosed region at risk (O_{z}) uses quantitative analysis of the volume of the stenosed region at risk (O_{z}) or percentage analysis in relation to the entire organ volume of the stenosed region at risk (O_{z}).

56. The system according to claim 50, wherein the module for superimposing uses the Voronoi diagram to visualise the ischaemic region (Oₙ).

57. A computer program embodied on a non-transitory computer readable medium for identifying heart ischaemia, comprising instructions for performing the method according to claim 38.

58. A product of execution by a computer processor of a computer program for identifying heart ischaemia, comprising a computer readable code embodied on a non-transitory computer readable medium performing the steps of the method according to claim 38.
